(19) United States
(12) Patent Application Publication
(10) Pub. No.: US 2011/0077518 A1
(43) Pub. Date: Mar. 31, 2011

(54) Ultrasonic diagnostic apparatus and method for calculating elasticity index

(75) Inventor: Yukiya Miyachi, Kanagawa (JP)

(73) Assignee: FUJIFILM Corporation, Tokyo (JP)

(21) Appl. No.: 12/923,401

(22) Filed: Sep. 20, 2010

(30) Foreign Application Priority Data
Sep. 28, 2009 (JP) 2009-223159

(51) Int. Cl. A61B 8/14

(52) U.S. Cl. 600/443

(57) ABSTRACT

An ultrasonic diagnostic apparatus is composed of an ultrasonic probe, a processor, and a monitor. The ultrasonic probe has multiple ultrasonic transducers arranged in one-dimension or two-dimensions. The adjacent L ultrasonic transducers are selected as a group. The group shifts in a state that the adjacent groups partly overlap with each other. The L ultrasonic transducers transmit and receive ultrasonic waves during the formation of the group. Multiple scan lines of echo data are acquired per transmission. The processor forms multiple frames of cross-sectional images for the same object of interest. A displacement amount of an organ is obtained using multiple frames. Based on an elasticity index calculated using the displacement amount, an elasticity index image in which the organ is colored is generated and displayed on a monitor in associated with a color map showing a relation between the elasticity index and the color.

### CROSS-REFERENCE TO RELATED APPLICATIONS

The present application claims priority from Japanese Patent Application No. 2009-223159, filed Sep. 28, 2009, the contents of which are herein incorporated by reference in their entirety.

### FIELD OF THE INVENTION

The present invention relates to an ultrasonic diagnostic apparatus which uses ultrasonic echo to observe living tissue, and more particularly to an ultrasonic diagnostic apparatus for calculating an elasticity index such as strain of a blood vessel wall or elastic modulus, and a method for calculating this elasticity index.

### BACKGROUND OF THE INVENTION

The number of patients suffering from cerebrovascular accidents, notably cerebral (brain) infarction and cerebral hemorrhage, or ischemic heart diseases such as myocardial infarction and angina pectoris are rapidly increasing. It is well known that most of the cerebrovascular accidents and the ischemic heart diseases result from arteriosclerosis. In order to prevent the cerebrovascular accidents and the ischemic heart diseases, it is essential to change or improve lifestyle to prevent the onset of the arteriosclerosis.

Conventional methods for noninvasive evaluation of artery such as measurements of PWV (pulse wave velocity) or ABI (ankle-brachial index) are known as examinations for arteriosclerosis. These methods use blood pressure measurements for evaluation of the artery, and provide general evaluation in which conditions of the artery in a large area are averaged.

The arteriosclerosis refers to thickening and stiffening of the blood vessel wall. A plaque is formed inside a part of a blood vessel wall as the arteriosclerosis advances, which reduces the inner diameter of the blood vessel. It is well known that a plaque rupture causes thrombus and embolus (blood clots) resulting in cerebrovascular accidents and ischemic heart diseases. To detect the plaque, an index to evaluate a local or topical area of the blood vessel wall is necessary in addition to that used to evaluate average condition of artery over a wide area. A method for measuring maximum intima-media thickness (abbreviated as IMT) of carotid artery, where the arteriosclerosis is likely to occur, has attracted attention.

Recently, in addition to the IMT, an index (hereinafter referred to as elasticity index) indicating elasticity or stiffness of the blood vessel wall, such as a stiffness parameter β, strain, a strain rate, or an elastic modulus, is used in arteriosclerosis examinations with displacement measurements of the blood vessel wall of the carotid artery in accordance with the cardiac cycle (see Japanese Patent No. 4091365 and U.S. Patent Application Publication No. 2004/0260180 corresponding to PCT Publication No. WO 03/015635). To obtain the elasticity index, it is necessary to precisely measure displacements of the blood vessel wall of the carotid artery, which moves in accordance with the cardiac cycle, while the blood vessel wall is tracked. An ultrasonic diagnostic apparatus capable of precisely tracking the blood vessel wall of the carotid artery is known (see Japanese Patent Laid-Open Publication No. 2006-325704). For the precise tracking, the ultrasonic diagnostic apparatus uses echo data obtained by emitting the ultrasonic beams to the carotid artery at two different angles.

To obtain an elasticity index with precision, the displacement amounts of the blood vessel wall and the IMT need to be measured in a direction passing the center of the blood vessel. An ultrasonic diagnostic apparatus which uses an ultrasonic probe (hereinafter referred to as 2D ultrasonic probe) having a 2-dimensional ultrasonic transducer array to obtain 3-dimensional data of the carotid artery and to measure the IMT and the like within a cross-section passing through the center of the carotid artery is known (see Japanese Patent Laid-Open Publication No. 2006-000456).

In addition, an ultrasonic diagnostic apparatus which obtains a cross-sectional image of the carotid artery with reciprocating motion of the ultrasonic probe to calculate the displacement amounts of the IMT in accordance with the cardiac cycle is known (see U.S. Patent Application Publication No. 2010/0121192 corresponding to Japanese Patent Laid-Open Publication No. 2008-237670).

As described above, the calculation of the elasticity index requires the tracking of the carotid artery wall with high precision. However, reproducible and reliable elasticity index cannot be calculated only with the highly precise tracking of the carotid artery wall. In addition, acquisition of echo data (or cross-sectional images generated from the echo data) at an appropriate rate relative to the displacement amount and the displacement speed of the carotid artery wall is required.

For example, strain ε of the carotid artery wall is expressed by an equation ε=ΔH/Hmax where Hmax represents the maximum thickness of the blood vessel wall within one cardiac cycle, ΔH represents a difference between the maximum thickness Hmax and the minimum thickness Hmin of the blood vessel wall within one cardiac cycle. In the case where an acquisition rate of the echo data is low relative to a displacement amount and a displacement speed of the carotid artery wall with reference to the cardiac cycle, the Hmax and the Hmin of the blood vessel wall cannot be calculated accurately even if the carotid artery wall is tracked with high precision in the acquired echo data. As a result, the strain ε becomes different in every calculation and thus it becomes difficult to secure reproducibility.

In the case where two echo data, acquired by emitting the ultrasonic beams from two directions, are used for the tracking of the carotid artery wall as described in Japanese Patent Laid-Open Publication No. 2006-325704, the acquisition rate of the echo data is substantially reduced by half. As a result, it becomes difficult to calculate an elasticity index with high reliability. Likewise, as described in Japanese Patent Laid-Open Publication No. 2006-000456, in the case where the IMT and the elasticity index are calculated after the acquisition of 3-dimensional data of the carotid artery using the 2D ultrasonic probe, it is necessary to acquire the echo data at a higher acquisition rate.

The IMT and the elasticity index need to be calculated with respect to the same point or area of a site. Accordingly, it is difficult to calculate the elasticity index with high reliability if the IMT values are calculated with respect to different points as described in Japanese Patent Laid-Open Publication No. 2006-000456.

### SUMMARY OF THE INVENTION

An object of the present invention is to provide an ultrasonic diagnostic apparatus and a method for calculating this elasticity index, capable of calculating an elasticity index with high reliability.

In order to achieve the above and other objects, the ultrasonic diagnostic apparatus of the present invention has multiple ultrasonic transducers, a transmitter, a receiver, an echo data generator, a memory, an elasticity index calculator, and an image generator. The ultrasonic transducers transmit ultrasonic beams to an object of interest and receive echo from the object of interest, and convert the received echo into echo signals. The transmitter forms L of the adjacent ultrasonic transducers into a group and inputs a drive signal to each of the ultrasonic transducers in the group to transmit the ultrasonic beams to the object of interest. The transmitter makes the ultrasonic transducers to transmit the ultrasonic beams with shifting the group by P (L>P≦2) ultrasonic transducers. The receiver receives the echo signals from the ultrasonic transducers. The echo data generator generates echo data for each scan line based on the echo signal. The echo data is data of the object of interest in a depth direction. The memory stores multiple frames of the echo data. An elasticity index calculator calculates an elasticity index of the object of interest based on the echo data. The image generator generates an elasticity index image. The elasticity index image is a cross-sectional image of the object of interest based on the echo data and colored in accordance with the elasticity index.

It is preferable that the ultrasonic diagnostic apparatus further includes a monitor for displaying the elasticity index image and a color map showing a relation between the elasticity index and a color.

It is preferable that a number N of transmission of the ultrasonic waves to acquire one frame of the echo data satisfies a mathematical expression (1) N≦(1/FR)×(Vs×10²/2D) where FR (unit: frame/s) represents a frame rate, Vs (unit: m/s) represents an average sound velocity (unit: m/s) in a human body, and D (unit: cm) represents a maximum depth of the object of interest.

It is preferable that the number N is determined to satisfy the mathematical expression (1) in accordance with a width and a depth of a region of interest when the region of interest in which the elasticity index is to be calculated is designated in the object of interest.

It is preferable that the number P is determined based on the number N. It is preferable that the frame rate FR is 100 or more. It is preferable that the one frame is produced with the number N of 58 or less.

It is preferable that the ultrasonic transducers are arranged in two dimensions. It is preferable that the echo data is generated for an area of at least 5 mm in azimuth direction by at least 5 mm in a lens direction, and it is preferable that the number N is 58 or less.

It is preferable that the object of interest is a blood vessel, and echo data relative to a cross-section passing through a center axis of the blood vessel is extracted along a center axis direction of the blood vessel, and the elasticity index inside the cross-section is calculated using the extracted echo data.

It is preferable that the object of interest is a blood vessel, and when the blood vessel moves, a displacement amount of the blood vessel is calculated using the echo data, and the elasticity index is calculated using the multiple frames of echo data of the same cross-section read from the memory based on the displacement amount.

It is preferable that the elasticity index is strain of the object of interest or a value calculated from the strain.

It is preferable that the elasticity index calculator determines multiple representative points in the depth direction of the object of interest in the echo data along at least one scan line in the frame. The elasticity index calculator identifies the representative points of the same scan line in another frame to track each representative point between the frames. Then a distance between the representative points is calculated for each frame, and strain between the representative points is calculated based on a maximum value of the calculated distance and a maximum change in the calculated distance.

The method for calculating an elasticity index of the present invention includes a transmitting step, a representative point determining step, an identifying step, a distance calculating step, and an elasticity index calculating step. In the transmitting step, ultrasonic waves are transmitted to an object of interest to obtain a first frame and a second frame sequentially. In the representative point determining step, multiple representative points are determined along at least one scan line in the first frame in the depth direction of the object of interest. In the identifying step, positions of the representative points are identified along the same scan line in the second frame to track each representative point between the first and second frames. In the distance calculating step, a distance between the representative points in each frame is calculated. In the elasticity index calculating step, strain of the object of interest between the representative points is calculated as the elasticity index based on a maximum value of the distance between the representative points and a maximum change in the distance between the representative points.

It is preferable that the method for calculating an elasticity index further includes an image producing step and a displaying step. In the image producing step, the first frame is colored in accordance with magnitude of the strain to produce an elasticity index image. In the displaying step, the elasticity index image and a color map are displayed on a monitor. The color map represents a relation between the magnitude of the strain and a color.

It is preferable that the object of interest is a blood vessel.

According to the present invention, the target site such as the carotid artery wall can be tracked with high precision while the echo data is acquired at high frame rate. Thus, the elasticity index is calculated with high reliability.

### BRIEF DESCRIPTION OF THE DRAWINGS

The above and other objects and advantages of the present invention will be more apparent from the following detailed description of the preferred embodiments when read in connection with the accompanied drawings, wherein like reference numerals designate like or corresponding parts throughout the several views, and wherein:

FIG. 1 is an explanatory view of a schematic configuration of an ultrasonic diagnostic apparatus;

FIG. 2 is a block diagram showing an electric configuration of the ultrasonic diagnostic apparatus;

FIG. 3 is an explanatory view showing observation of a carotid artery;

FIGS. 4A and 4B are explanatory views showing ultrasonic beams;

FIGS. 5A, 5B, and 5C are explanatory views showing generation of echo data from a receive signal using an area former;

FIGS. 6A and 6B are explanatory views showing cross-sectional images;

FIGS. 7A, 7B, and 7C are explanatory views showing calculation of strain;

FIGS. 8A, 8B, and 8C are explanatory views showing generation and display of a strain image;

FIGS. 9A, 9B, and 9C are explanatory views showing calculation of strain in a specified ROI;

FIGS. 10A and 10B are explanatory views showing transmission of ultrasonic beams using a 2D ultrasonic probe; and

FIGS. 11A to 11D are explanatory views showing the observation of the carotid artery using the 2D ultrasonic probe.

### DESCRIPTION OF THE PREFERRED EMBODIMENTS

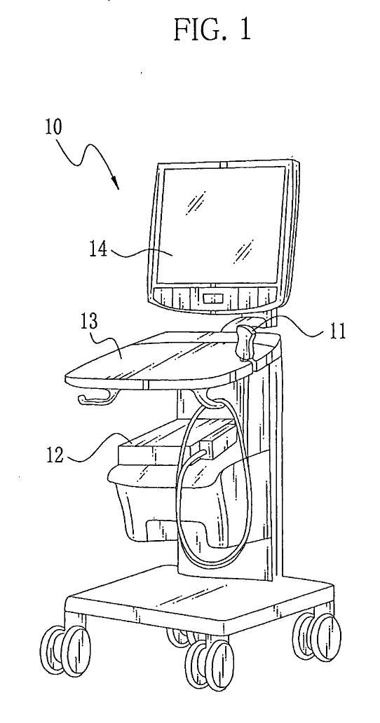

As shown in FIG. 1, an ultrasonic diagnostic apparatus 10 is composed of an ultrasonic probe 11 and a processor 12. The ultrasonic probe 11 transmits ultrasonic waves to the inside of the body of a patient and receives echo waves. The processor 12 forms an image of the inside of the body based on the echo waves. The image is displayed on a monitor 14 as an ultrasonic cross-sectional image. An operation section 13 is connected to the ultrasonic diagnostic apparatus 10.

The ultrasonic probe 11 is provided with plurality of ultrasonic transducers 16 (see FIG. 2) arranged along its tip. Each ultrasonic transducer 16 transmits and receives ultrasonic waves. When in use, the tip of the ultrasonic probe 11 is contacted on the body surface of the patient. The ultrasonic probe 11 is connected to the processor 12 via a cable.

The processor 12 controls overall operations of the ultrasonic diagnostic apparatus 10. The processor 12 causes the ultrasonic probe 11 to transmit and receive ultrasonic waves. The processor 12 generates cross-sectional images such as B mode images and M mode images from the received echo waves to display them on the monitor 14. The processor 12 calculates elasticity index such as strain and elastic modulus of living tissue being observed. The processor 12 generates a strain image, that is, an image colored according to such elasticity index, and displays it on the monitor 14 real time. Alternatively or in addition, the elasticity index may be expressed on a gray scale.

The operation section 13 is composed of a key board, a pointing device, various buttons, a dial, and the like. An operator such as a doctor operates the ultrasonic diagnostic apparatus 10 through the operation section 13. Using the operation section, the operator specifies various setting values related to the operation of the ultrasonic diagnostic apparatus 10 in accordance with an object of interest (living tissue) and changes a focal depth of ultrasonic beams transmitted from the ultrasonic probe 11, for example. The operator specifies a region of interest (hereinafter abbreviated as ROI) using the operation section 13. Data obtained by other devices such as blood pressure measurements of the patient are input using the operation section 13.

As shown in FIG. 2, the ultrasonic transducers 16 are arranged in one line in the ultrasonic probe 11. Each ultrasonic transducer 16 is composed of a piezoelectric element. Each ultrasonic transducer 16 is pulse-driven by a drive signal input from a transmitter 22 through a multiplexer. For example, to transmit ultrasonic beams from the ultrasonic probe 11, successive or adjacent L (L≧4) ultrasonic transducers 16 are selected as a group from among all the ultrasonic transducers 16, and each ultrasonic transducer in the group is driven with a delay. Thereby, the L ultrasonic transducers 16 sequentially transmit the ultrasonic waves to a target area of the tissue, which converge into wide ultrasonic beams. Upon incident of echo of the ultrasonic beams on the ultrasonic transducers 16 contained in the group, each ultrasonic transducer 16 outputs an analog echo signal in accordance with amplitude of the incident echo. The echo signals output from the ultrasonic transducers 16 are input to a receiver 23 through the multiplexer 21.

The processor 12 is composed of a multiplexer 21, the transmitter 22, the receiver 23, a quadrature detection section 24, a memory 26, an area former 27, an image generator 28, an elasticity index calculator 29, a controller 30 and the like.

The multiplexer 21 selectively inputs a drive signal output from the transmitter 22 to each of the L ultrasonic transducers 16 to form a group of actuatable ultrasonic transducers 16. The multiplexer 21 individually inputs an echo signal output from the L ultrasonic transducers 16 to the receiver 23.

The transmitter 22 generates drive signals to cause the ultrasonic transducers 16 to transmit ultrasonic waves. The drive signal is input to each of the L ultrasonic transducers 16 contained in the group from among all the ultrasonic transducers 16 to pulse-drive the L ultrasonic transducer 16. The drive signal is input to each of the L ultrasonic transducers 16 with a delay depending on the shape, the focal depth, the size, and the like of the ultrasonic beams transmitted from the ultrasonic probe 11. The transmitter 22 sequentially shifts or changes the group (the L actuatable ultrasonic transducers 16) by a predetermined pitch or shift amount P (P≧2) in one direction to transmit ultrasonic beams intermittently. The pitch P is determined based on the number N of the transmission of the ultrasonic beams per frame. The pitch P is determined such that two successive transmissions of the ultrasonic beams are partly overlapped with each other with at least one scan line of data being obtained twice. Here, the L ultrasonic transducers 16 in the group to be driven per transmission of the ultrasonic beams is determined such that one scan line is overlapped during the two successive transmissions when the subsequent group to be driven is shifted or changed by the pitch P. In this embodiment, L is seven, and P is two.

The receiver 23 receives the analog echo signals output from the ultrasonic transducers 16 through the multiplexer 21, and then inputs them to the quadrature detection section 24. The receiver 23 amplifies the analog echo signals, and then converts them into digital data.

The quadrature detection section 24 multiplies the digital echo signal input from the receiver 23 by a sine wave, and by a cosine wave both having the same frequency as the center frequency of the ultrasonic waves. Then, each of the digital echo signals are passed through a low pass filter. Thus, the digital echo signals are converted into complex baseband signals containing information of amplitude and phase. The complex baseband signals output from the quadrature detection section 24 are temporarily stored in the memory 26, and then used by the area former 27.

The area former 27 reads multiple complex baseband signals from the memory 26, and performs phase matching and addition of the complex baseband signals. Thus, data of the object of interest in a depth direction (hereinafter referred to as echo data) is generated for each scan line. Multiple frames of generated echo data are stored in the memory 26. The stored echo data is used by the image generator 28 and the elasticity index calculator 29.

The image generator 28 generates a cross-sectional image based on a series of the predetermined number of echo data. For example, the image generator 28 generates a B-mode image from one frame of echo data. The image generator 28 generates an M-mode image from the echo data of the same scan line from among temporally successive multiple frames of echo data. The image generator 28 generates an elasticity index image which displays an elasticity index calculated by the elasticity index calculator 29. Based on the elasticity index value calculated for each area of the object of interest, each area in a cross-sectional image, such as a B mode image, is colored according to a color map. Thus, various cross-sectional images generated by the image generator 28 are displayed on the monitor 14 together with the color map.

The elasticity index calculator 29 calculates strain ε per subdivided area inside the object of interest with the use of multiple frames of echo data, for example. First, the elasticity index calculator 29 determines representative points in each scan line based on properties of a waveform of the echo data of a selected frame. Next, in the predetermined number of frames of echo data, a position of each representative point is identified per scan line in each frame using pattern matching based on the phase and the amplitude of the echo data. Thus, each representative point is tracked across the multiple frames. Thereafter, a distance between the adjacent representative points is calculated for each frame. The maximum distance ha between the representative points and the minimum distance hb between the representative points are calculated. A difference between the maximum distance ha and the minimum distance hb is calculated. A maximum change Δh in the distance between the representative points is calculated using an equation Δh=ha−hb. The elasticity index calculator 29 calculates the strain ε of the tissue between the representative points using the equation ε=Δh/ha, based on the maximum distance ha between the representative points, and the maximum change Δh in the distance between the representative points. In the case where the operator designates an ROI, the elasticity index calculator 29 calculates the strain ε for an area inside the ROI in the same manner as the above. The strain ε calculated by the elasticity index calculator 29 is stored in the memory 26 in associated with the position information of each of the representative points. The image generator 28 uses the stored strain ε and the position information to generate an elasticity index image.

Upon the input from the operation section 13, the controller 30 controls each section of the processor 12. The controller 30 decides the number N of transmission of ultrasonic beams to satisfy the following mathematical expression (1) where “N” represents the number of transmission of ultrasonic beams for acquisition of the echo data of one frame, “FR” represents a frame rate (unit: frame/s), “Vs” represents an average sound velocity (unit: m/s) inside a human body, “D” represents a maximum depth (unit: cm) of living tissue whose elasticity index is to be calculated. It is preferable that the number N of transmission of the ultrasonic beams be the largest integer satisfying the mathematical expression (1).

N≦1/FR×(Vs×10²)/2D (1)

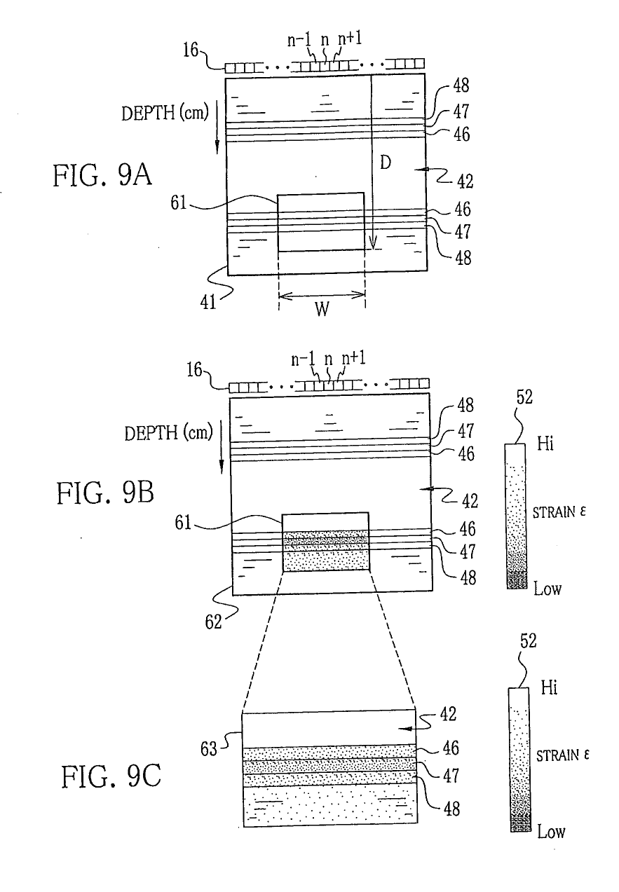

The mathematical expression (1) describes the following. The average sound velocity Vs inside the human body is approximately in a range from 1400 (m/s) to 1600 (m/s), and regarded as substantially constant. For this reason, the average sound velocity is fixed to a value within the above range at the start of the use of the ultrasonic diagnostic apparatus 10, for example. A time required for the ultrasonic waves to travel to the target and then return to the ultrasonic transducers 16 is expressed as “2D/(Vs×10²)” where D represents the depth of the target, for example, in this case, the depth of the deepest area of the carotid artery. When a region of interest (ROI) is not designated, the controller 30 uses the depth of the bottom or the lowest edge of the cross-sectional image as “D”. When an ROI is designated, the distance between the ultrasonic transducers 16 and the deepest area of the ROI is used as “D” (see FIGS. 9A and 9B).

On the other hand, to calculate the strain ε with good reproducibility, it is necessary to obtain the echo data for “M” frames of B mode images within “T” seconds (for example, seconds of one cardiac cycle). To obtain one frame (that is, one piece of B image) of the echo data, the ultrasonic beams are transmitted “N” times. Accordingly, the ultrasonic beams needs to be transmitted N×M times within a predetermined time T.

The time required for the ultrasonic waves to travel to the target and then return to the ultrasonic transducers 16 is defined by the mathematical expression 2D/(Vs×10²) as described above. Accordingly, to acquire echo data in a predetermined time T (second), the ultrasonic beams can be transmitted T×(Vs×10²)/2D times at the maximum. Therefore, N×M is equal to or less than T×(Vs×10²)/2D. The number of transmission N needs to satisfy N≦(T/M)×(Vs×10²)/2D. Here, T/M=1/(M/T), and M/T is the frame rate FR. Thus, the above mathematical expression (1) is obtained. If the number N of transmission of ultrasonic waves is larger than the right side of the mathematical expression (1), the quantity of the echo data acquired within the time T becomes small. The strain ε calculated based on such echo data is poor in reproducibility. As a result, the reliability of the strain ε is reduced.

A pitch P to shift or change the group of actuatable ultrasonic transducers 16 is determined based on the number N of transmission of ultrasonic beams, a width of echo data per scan line, and a width of a cross-sectional image to be generated. For example, in the case where N=58, and the echo data of one scan line generates a cross-sectional image having 0.3 mm in width, and in total a cross-sectional image having 33 mm in width is to be generated, the pitch P is determined as a minimum integer larger than a value obtained using the mathematical expression: (33 ram/0.3 mm)÷58≈1.9. In this case, P is two.

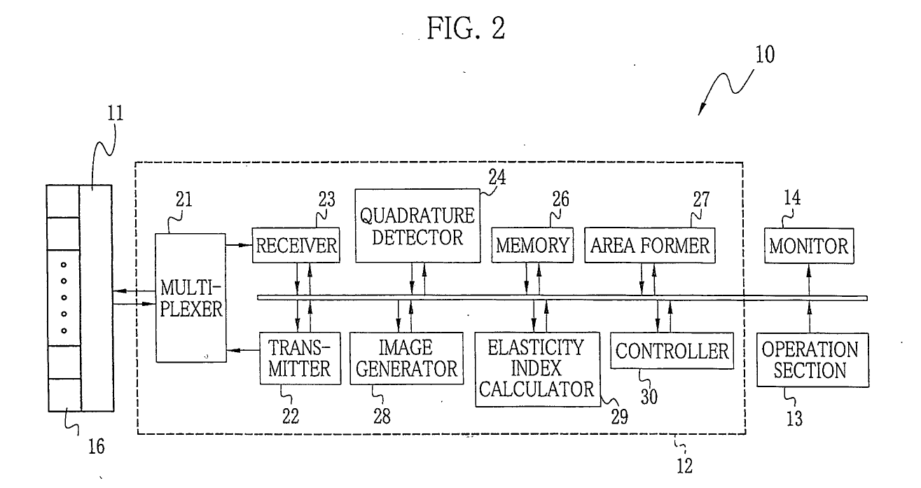

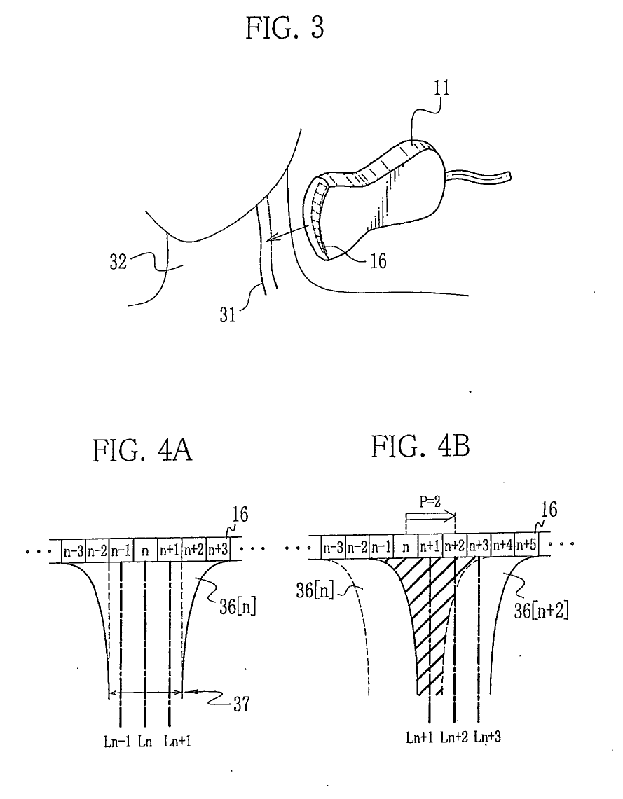

Hereinafter, as shown in FIG. 3, an operation of the ultrasonic diagnostic apparatus 10 is described with an example in which a carotid artery wall is observed while the ultrasonic probe 11 is contacted on a neck 32 of a patient along a carotid artery 31. Generally, a depth of the carotid artery 31 is in a range from 2 cm to 4 cm, so a depth D required for the observation is approximately 3 cm. The average speed Vs of sound within a human body is in a range from 1400 m/s to 1600 m/s. It is known that the carotid artery wall of a healthy individual moves approximately 0.5 mm within one cardiac cycle and its moving speed is approximately in a range from 5 mm/s to 8 mm/s. To calculate strains with high reproducibility and high reliability, accurate tracking is necessary. The measurement errors of the representative points need to be reduced to at most 10% of the displacement amount of the carotid artery wall (0.5 mm): A frame rate FR for the observation of the carotid artery of a healthy individual is calculated as follows. 0.05 (mm)÷5 (mm/s)=0.01 (s). It means that the frame rate FR needs to be at least 100 (frame/s) to track the displacement of 0.05 mm. For the patient with the blood vessel wall moving faster than the above per cardiac cycle due to high blood pressure or the like, the FR is preferred to be approximately 400 (frame/s). In consideration of the above, D=3, Vs=1400, and FR=400 are substituted into the mathematical expression (1), and the number N of transmission of ultrasonic beams per frame is specified as “58”.

The ultrasonic diagnostic apparatus 10 generates the echo data of 110 scan lines. For each line, the ultrasonic diagnostic apparatus 10 generates an image corresponding to an area of 0.3 mm in width. Accordingly, a width of a B mode image generated by the ultrasonic diagnostic apparatus 10 becomes 33 mm. As described above, since the number N of the transmission of the ultrasonic beams per frame is specified as 58, the echo data of at least two scan lines needs to be generated per transmission of the ultrasonic beams. Accordingly, the pitch P of the group is determined to two.

When the observation of the carotid artery 31 is started using the ultrasonic diagnostic apparatus 10, the transmitter 22 intermittently transmits wide ultrasonic beams inside the body of the patient. For example, as shown in FIG. 4A, the total of seven adjacent ultrasonic transducers 16, from (n−3)thh to (n+3)thh ultrasonic transducers 16, are driven, each with a delay, to transmit the wide ultrasonic beams 36[n] such that the transmitted ultrasonic waves converge into a range of three adjacent ultrasonic transducers 16 from (n−1)thh to (n+1)thh ultrasonic transducers 16. The number “n” is counted from an end of the ultrasonic transducers 16 arranged in one line. A focal zone 37 of the ultrasonic beams 36[n] is determined at a depth where the width of the converged ultrasonic beams 36[n] becomes approximately the same as the total width of three adjacent ultrasonic transducers 16 from the (n−1)thh to the (n+1)thh ultrasonic transducers 16. Mainly, the echo from tissue close to the focal zone 37 is received by the ultrasonic transducers 16. In other words, the tissue close to the focal zone 37 is clearly observed. Since the depth of the carotid artery is approximately 2 cm to 4 cm, the depth of the focal zone 37 is previously set to 3 cm.

The echo of the ultrasonic beams 36[n] is received with all the ultrasonic transducers 16. However, the receiver 23 selectively receives the echo signals from the seven ultrasonic transducers 16 used for the transmission of the ultrasonic beams, namely, from (n−3)thh to (n+3)thh ultrasonic transducers 16. The area former 27 generates the echo data of scan lines Ln−1, Ln, and Ln+1, corresponding to (n−1)thh, nth, and (n+1)thh ultrasonic transducers 16, respectively, based on the echo signal converted into the complex baseband signal.

As shown in FIG. 4B, the pitch P is set to two, for example. The group is shifted from the precedingly driven group at the pitch or interval of two ultrasonic transducers 16. The group consists of seven ultrasonic transducers 16 from the (n−1)thh to (n+5)thh ultrasonic transducers 16 with the (n+2)thh ultrasonic transducer 16 as the center of the group. Each of the seven ultrasonic transducers 16 in the group are driven sequentially with a predetermined delay. The ultrasonic beams transmitted to the inside of the object of interest are converged into wide ultrasonic beams 36[n+2] having the width of 3 ultrasonic transducers 16, from the (n+1)thh to (n+3)thh ultrasonic transducers 16. The receiver 23 selectively receives echo signals from seven ultrasonic transducers 16, from the (n−3)thh to (n+3)thh ultrasonic transducers 16, in the same manner as the reception of the ultrasonic beams 36[n]. Based on the echo signals modulated into the complex baseband signals, the area former 27 generates echo data of the scan line corresponding to the ultrasonic transducer 16, for example, of the scan lines Ln+1, Ln+2, and Ln+3 corresponding to the (n+1)thh, (n+2)thh, and (n+3)thh ultrasonic transducers 16, respectively.

The ultrasonic probe 11 repeats the transmission of wide ultrasonic beams, and thus the scanning of the object of interest is performed across the width equivalent to that of the ultrasonic transducers 16. The transmitter 22 transmits the ultrasonic beams 36[n+2] such that the ultrasonic beams 36[n+2] and the ultrasonic beams 36[n] partly overlap with each other as shown in a hatched area in FIG. 4B. Thereby, the ultrasonic diagnostic apparatus 10 generates three scan lines of echo data per transmission of ultrasonic beams. In each transmission, one line of echo data is overlapped, and two scan lines of new echo data are generated. On the other hand, a common ultrasonic diagnostic apparatus generates one line of echo data per transmission of ultrasonic beams. When the wide ultrasonic beams are transmitted at a pitch P (P=2) to shift or change the group (consisting of seven ultrasonic transducers) to be driven, the ultrasonic diagnostic apparatus 10 acquires the echo data at a rate approximately P times (in this case two times) higher than that of the common ultrasonic diagnostic apparatus. The overlapping echo data is used for registration of two scan lines of new echo data generated per transmission.

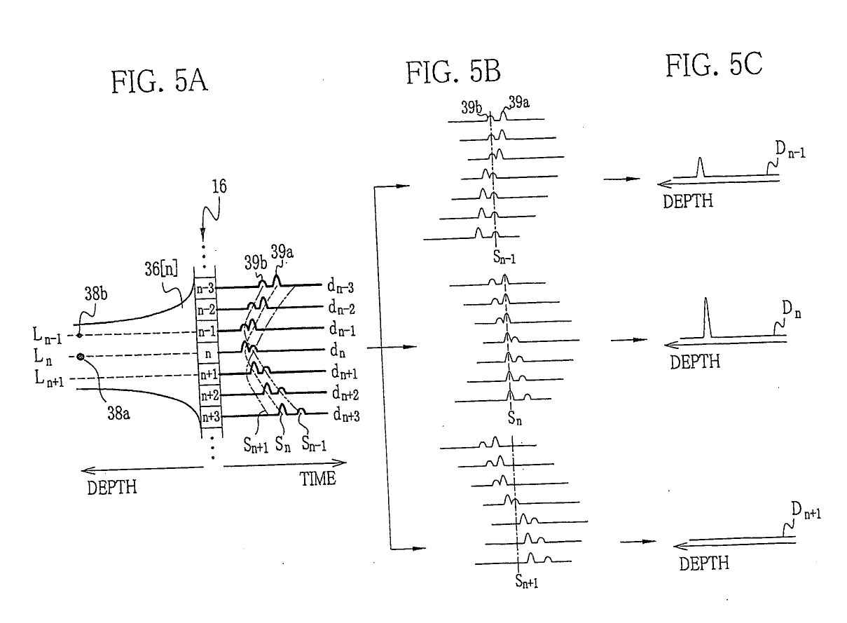

As described above, after the transmission of the ultrasonic beams by the group of seven ultrasonic transducers 16, the three scan lines of the echo data are generated based on the received seven pieces of the echo data. For example, as shown in FIG. 5A, when the ultrasonic beams 36[n] are transmitted, strong scatterers 38a and 38b exist on the scan line Ln−1 and the scan line Ln, respectively. In each of the selectively received echo data dn−3 to dn+3, amplitudes of signals 39a and 39b appear. However, distances between the scatterer 38a and each of the (n−3)thh to (n+3)thh ultrasonic transducers 16 differ from each other, and distances between the scatterer 38b and each of the (n−3)thh to (n+3)thh ultrasonic transducers 16 differ from each other. As a result, the signals 39a and 39b appear in different positions in each of the echo signals dn−3 to dn+3 in accordance with the distances between the ultrasonic transducer 16 and each of the scatterers 38a and 38b.

As shown in FIG. 5B, the area former 27 performs phase matching to the echo signals dn−3 to dn+3 converted into the complex baseband signals, and then adds the signals. In the phase matching, the positions of the signals Sn−1 from the same point are matched, and the positions of the signals Sn from the same point are matched, and the positions of the signals Sn+1 from the same point are matched. Thus, the echo data Dn−1, Dn, and Dn+1 is generated in accordance with the scan lines Ln−1, Ln, and Ln+1 in the depth direction, respectively. For example, when the echo data of the scan line Ln is generated, the echo signals dn−3 to dn+3 are added while they are shifted in a time direction such that the positions of the signals Sn, from the same point on the scan line Ln, match. Thus, as shown in FIG. 5C, on the echo data Dn, signals such as the signals 39b from the scatterer 38b on the scan line Ln−1 are averaged to a noise level. Only a signal in which the signal 39a from the scatterer 38a on the scan line Ln is enhanced appears on the echo data Dn. Likewise, on the echo data Dn−1 and Dn+1, only enhanced signals from the scatterers on the scan lines Ln−1, and Ln+1 appear, respectively.

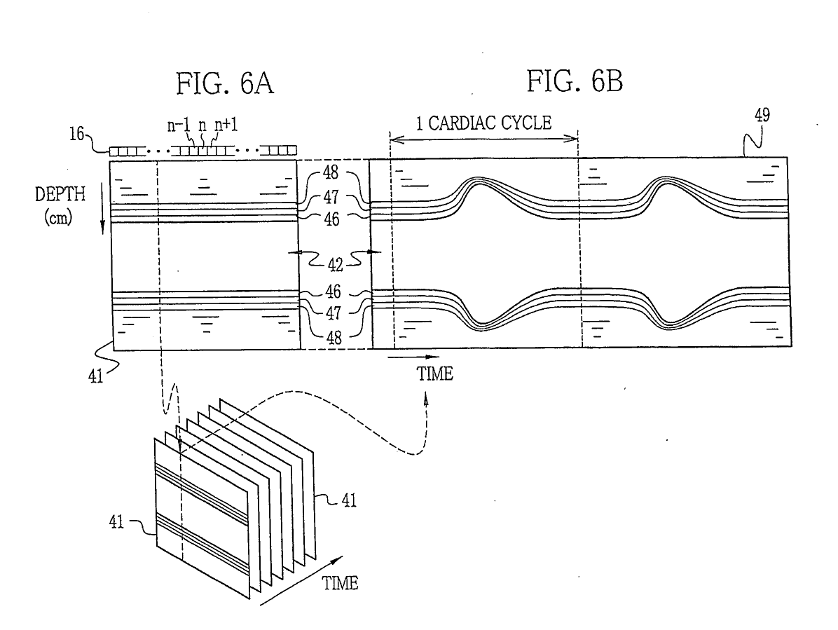

As shown in FIG. 6A, the image generator 28 arranges the echo data generated as described above, and maps the amplitude of each echo data as brightness. Thus, the image generator 28 generates a B mode image. The B mode image shows a cross-section of the carotid artery 31 on a gray scale. For example, the B mode image 41 shows a cross-sectional layer structure of the carotid artery 31 composed of tissue like lumen 42, tunica intima 46, tunica media 47, and tunica adventitia 48.

As shown in FIG. 6B, the image generator 28 selects the echo data of one scan line, for example, the scan line Ln, from among the echo data corresponding to temporally-successive frames of B mode image 41. The selected echo data is arranged in time order with a predetermined width. The amplitude of each echo data is mapped as brightness. Thus, an M mode image 49 is generated. The M mode image 49 shows changes in the carotid artery 31 with time. For example, the M mode image 49 shows that each of tissue 42, and 46 to 48 displaces in the depth direction with the cardiac cycle as the carotid artery 31 repeats the dilation and contraction.

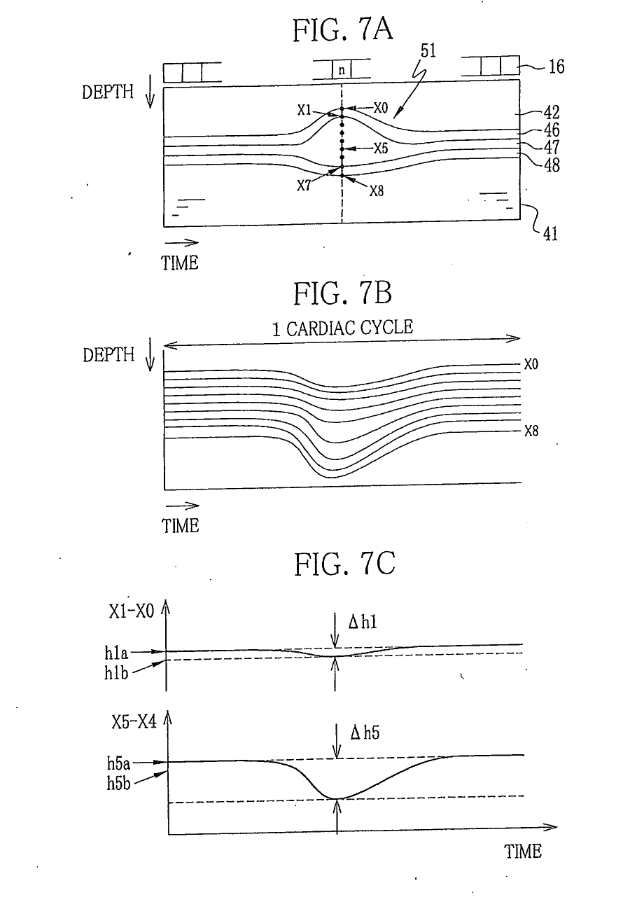

As described above, the image generator 28 generates the cross-sectional image, and the elasticity index calculator 29 calculates the strain £ of each tissue of the carotid artery 31 based on the multiple frames of echo data. For convenience in explanation, as shown in FIG. 7A, for example, an area with a plaque 51 is observed. The elasticity index calculator 29 reads echo data of a certain frame on a line-by-line basis to determine representative points based on the phase and amplitude of the echo data. For example, the elasticity index calculator 29 determines representative points X0 to X8 on the echo data of the scan line Ln. The point X0 is located at the interface between the lumen 42 and the tunica intima 46. The point X1 is located at the interface between the tunica intima 46 and the tunica media 47. The five subsequent representative points are located inside the tunica media 47. The point X7 is located at the interface between the tunica media 47 and the tunica adventitia 48. The point X8 is located at the interface between the tunica adventitia 48 and tissue outside the carotid artery 31.

Next, as shown in FIG. 7B, multiple frames of echo data each having the representative points X0 to X8 are subjected to pattern matching. The representative points X0 to X8 are identified on the scan line Ln in each frame. Thereby, tracking data in which depths of the representative points X0 to X8 are tracked in time order is temporarily generated. Based on the tracking data, the elasticity index calculator 29 calculates a distance between the adjacent representative points within one cardiac cycle. Then, the elasticity index calculator 29 acquires the maximum value, and the minimum value of the calculated distance, and the maximum change between the maximum and minimum values. Based on these values, the strain ε of the tissue between the representative points is calculated.

For example, as shown in FIG. 7C, distances between the representative points X1 and X0 are calculated within one cardiac cycle to obtain the maximum value h1a and the minimum value h1b thereof. A maximum change Δh1(=h1a−h1b) in the distance between the representative points X1 and X0 is calculated. Then, strain ε1(=Δh1/h1a) is calculated. The calculated strain ε1 represents strain of the tunica intima 46 at the top of the plaque 51 and corresponds to the cardiac cycle.

For example, distances between the representative points X5 and X4 are calculated within one cardiac cycle to obtain the maximum value h5a and the minimum value h5b thereof. A maximum change Δh5 (=h5a−h5b) in the distance between the representative points X5 and X4 is calculated. Then, strain ε5(=Δh5/h5a) is calculated. The calculated strain ε5 represents strain inside the plaque 51 and corresponds to the cardiac cycle.

Likewise, the elasticity index calculator 29 calculates distances between remaining representative points to obtain the maximum value and the minimum value thereof. A maximum change in the distance between the representative points is calculated. Then, strain ε is calculated using the calculated values. Here, the echo data of the scan line Ln is described as an example. The elasticity index calculator 29 calculates the strain ε for the tissue on all scan lines in the same manner as described above.

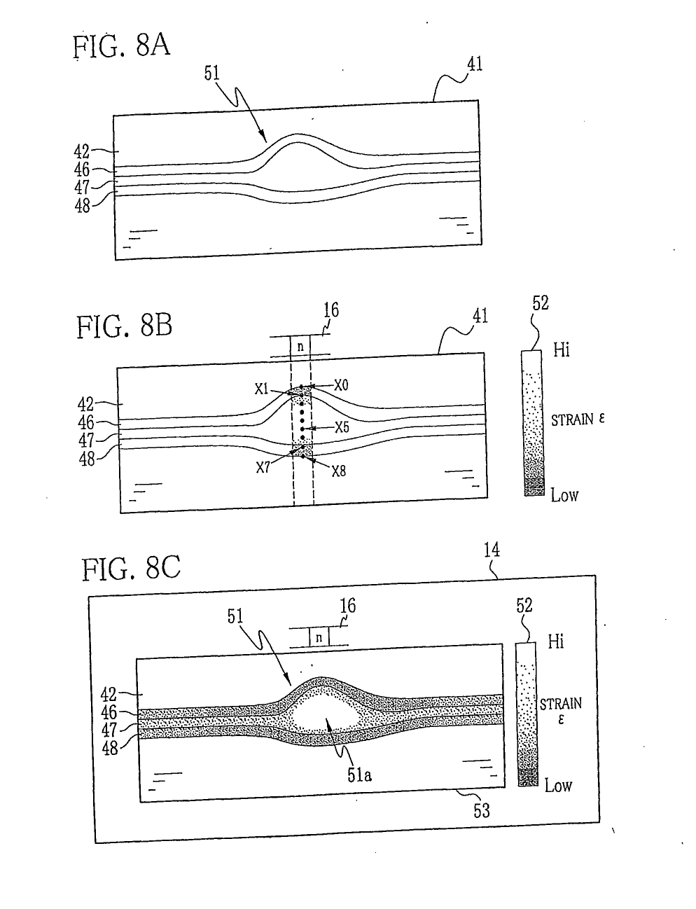

After the strain ε is calculated by the elasticity index calculator 29, the image generator 28 colors the B mode image 41 of the blood vessel wall shown in FIG. 8A according to the color map indicating the magnitude of the strain ε. As shown in FIG. 8B, for example, a pixel on the scan line Ln is partitioned into areas by the representative points X0 to X8, and each area between the representative points is colored using a color map 52. In FIG. 8B, the color map 52 is shown on a gray scale for the sake of convenience. Actually, for example, the area with the high strain ε is colored with blue, and the color gets still more blue as the strain ε increases. On the contrary, the area with the low strain ε is colored with red, and the color gets still more red as the strain ε decreases. Likewise, the image generator 28 colors the whole B mode image 41 on a line-by-line basis. Thus, an elasticity index image 53 is generated. As shown in FIG. 8C, the generated elasticity index image 53 and the color map 52 are arranged side by side on the monitor 14. The elasticity index image 53 shows a cross-sectional wall structure of the carotid artery 31 and the magnitude of the strain ε in each tissue of the carotid artery wall. The elasticity index image 53 shows, inside the plaque 51, a soft tissue 51a such as lipid having larger strain ε than the surrounding tissue, which cannot be discriminated in the B mode image 41.

As described above, the ultrasonic diagnostic apparatus 10 transmits the wide ultrasonic beams to generate the echo data of multiple scan lines per transmission. For the subsequent transmission of ultrasonic beams, the group of the ultrasonic transducers 16 to be driven is shifted or changed by two or more ultrasonic transducers. Thereby, one frame of the echo data is obtained at high speed to achieve a frame rate as high as FR=400 when compared to a so-called line-by-line acquisition in which one line of echo data is generated per transmission. As a result, the strain ε is calculated with high reproducibility and high reliability.

Since the number N of transmission of ultrasonic beams per frame satisfies the mathematical expression (1), multiple lines of echo data required for generating a cross-sectional image with high resolution are acquired at the above described high frame rate.

In the above embodiment, the strain ε is calculated for one frame of the whole B mode image 41 to generate the elasticity index image 53. However, it is preferable to calculate strain ε for a designated part of the B mode image 41. For example, as shown in FIG. 9A, an operator designates an ROI 61 while observing the B mode image 41. The ultrasonic diagnostic apparatus 10 changes the number N of transmission of ultrasonic beams per frame to a maximum integer satisfying the mathematical expression (1) where “D” represents the distance between the ultrasonic transducer and the deepest area of the ROI 61. As shown in FIG. 9B, instead of calculating the strain ε for the whole B mode image 41, the ultrasonic diagnostic apparatus 10 calculates the strain ε inside the designated ROI 61 in the same manner as the above. In this case, the color map 52 and an elasticity index image 62 of the ROI 61 colored according to the color map 52 are displayed on the monitor 14.

In FIG. 9B, tissue around the ROI 61 is displayed. Alternatively, as shown in FIG. 9C, an elasticity index image 63 only showing the enlarged ROI 61 may be generated, and displayed on the monitor 14. When the strain ε inside the designated ROI 61 is calculated to generate the elasticity index image 63 of the ROI 61, it is preferable to generate the echo data of the ROI 61 only. It is preferable that the ultrasonic diagnostic apparatus 10 determines the number N of transmission of the ultrasonic beams to satisfy the mathematical expression (1) in accordance with a width W of the ROI 61 and the number of scan lines (in this case, the number of the ultrasonic transducers 16) contained in the width W. It is preferable to change the pitch P in accordance with the number N of the transmission and the width W of the ROI 61. Thus, the strain ε is calculated with high reproducibility and high reliability, and displayed as in the above embodiment.

The size of the early lesion of arteriosclerosis is in a range approximately from 1 mm to 10 mm. To observe the carotid artery 31 as described in the above embodiment, it is preferable to generate echo data for an area having a width of at least 5 mm. Even if the designated ROI is smaller than this, it is preferable to generate the echo data for the area having a width of at least 5 mm and containing the designated ROI.

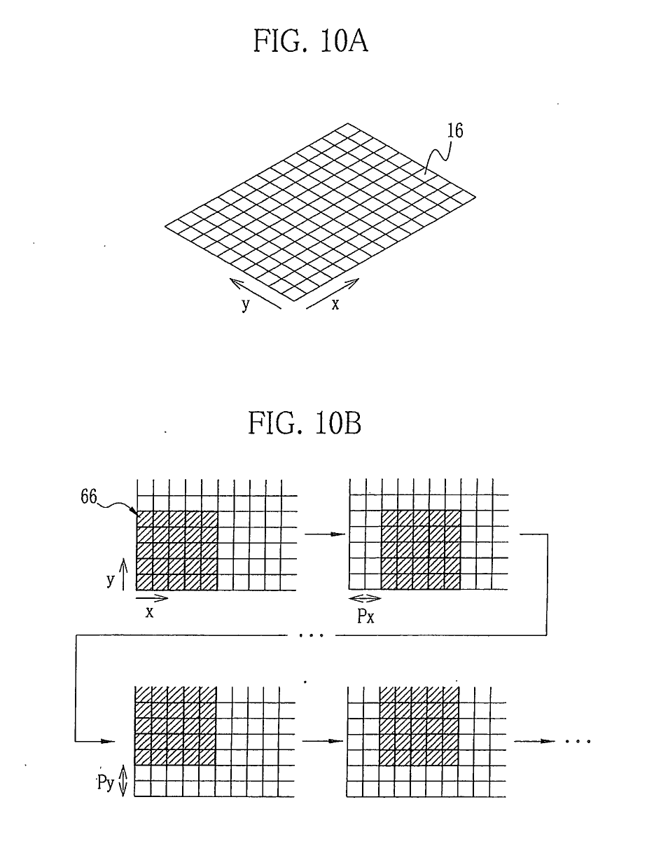

In the above embodiment, the ultrasonic transducers 16 are arranged in one line as an example. Alternatively, as shown in FIG. 10A, a 2-dimensional ultrasonic probe (hereinafter referred to as 2D ultrasonic probe) having the ultrasonic transducers 16 arranged in 2 dimensions can be used. With the use of the 2D ultrasonic probe, the high frame rate is achieved, and the strain ε is calculated with high reproducibility and high reliability as in the above embodiment. In this case, for example, as shown in hatched areas in FIG. 10B, the echo data is acquired with a group 66 (5×5, a total of 25 ultrasonic transducers 16) to be driven per transmission of ultrasonic beams with shifting or changing the group 66 by a pitch Px in x direction. When the scanning is completed to one of the ends, the group 66 is shifted or changed by a pitch Py in y direction, and then again the echo data is acquired with the group 66 with shifting or changing the group 66 by the pitch Px in the x direction. The above processes are repeated. Thus, the 2D ultrasonic probe achieves a high frame rate which cannot be achieved by the line-by-line acquisition and acquires echo data required for the calculation of a strain ε with high reliability.

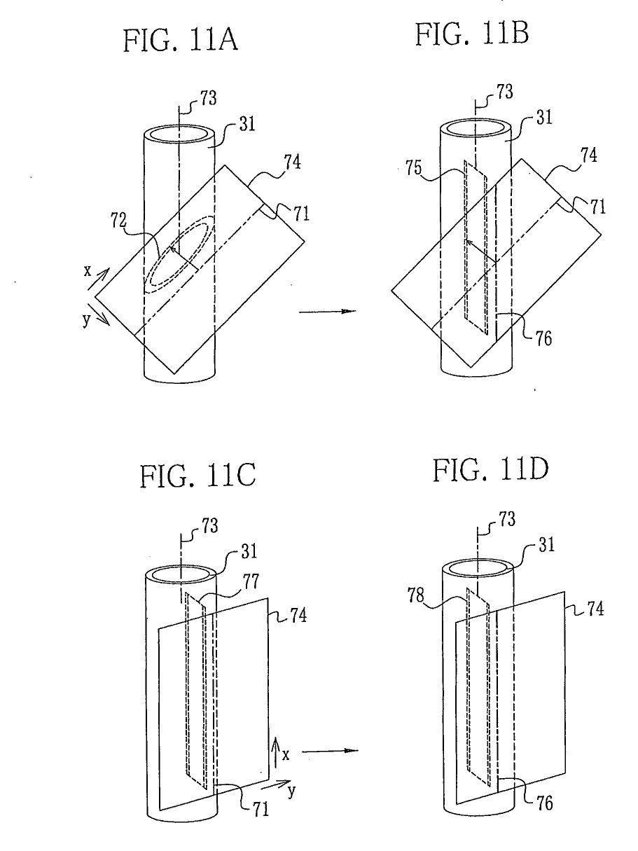

The 2D ultrasonic probe provides a strain ε with higher reproducibility and higher reliability when compared to an ultrasonic probe provided with the ultrasonic transducers 16 arranged in one line (hereinafter may referred to as 1D ultrasonic probe). For example, as shown in FIG. 11A, the ultrasonic probe may be contacted at a certain angle relative to the carotid artery 31. In this case, when the 1D ultrasonic probe is used, a cross-section of the carotid artery 31 along the line 71, namely, an oval cross-section 72 is observed. However, the strain ε has the highest reproducibility and the highest reliability when it is calculated using echo data of a scan line passing a center axis 73 of the carotid artery 31. As the scan line becomes away from the center axis 73, the obtained strain ε has a larger error from the actual strain ε. The 2D ultrasonic probe, on the other hand, acquires 3-dimensional echo data on all scan lines on a plane 74. For example, as shown in FIG. 11B, the 2D ultrasonic probe extracts echo data of a cross-section 75 passing the center axis 73 of the carotid artery 31 along a line 76. The line 76 is a line to which a cross-section vertical to the plane 74 is projected, and along which the diameter of the carotid artery is at the maximum. The strain ε is calculated based on this echo data. Thus, the strain ε with high reproducibility and high reliability is constantly calculated.

As shown in FIG. 11C, the ultrasonic probe may be contacted to a patient with its center displaced to the side from the center of the carotid artery 31. In this case, when the 1D ultrasonic probe is used, only a cross-section 77 along a line 71 displaced from the center axis 73 of the carotid artery 31 can be observed. As a result, the reliability of the strain ε decreases when compared to the calculation using echo data of the scan line passing the center axis 73. On the other hand, as shown in FIG. 11D, the 2D ultrasonic probe extracts the echo data of a cross-section 78 passing the center axis 73 from the acquired 3-dimensional echo data. With the use of the 2D ultrasonic probe, the strain ε with high reproducibility and high reliability is constantly calculated.

During the observation of the carotid artery 31, the carotid artery 31 may be displaced due to heartbeats, motion of the patient, motion of a hand of the operator, or the like. Since the 2D ultrasonic probe covers a wide range, there is a high possibility of scanning the cross-sections 75 and 78. As a result, the strain ε with the high reproducibility and high reliability is calculated with more ease. For example, when the carotid artery 31 is 3-dimensionally displaced, a displacement amount of the carotid artery 31 (for example, the displacement amount of the center axis thereof) is calculated based on the 3-dimensional echo data acquired prior to and after the displacement of the carotid artery 31. Based on the calculation, the echo data of the same cross-section of the carotid artery 31 is extracted from a frame of the echo data acquired after the displacement. The strain ε is calculated using the extracted echo data. For the use of 2D ultrasonic probe, it is necessary to provide a circuit or the like specific to the 2D ultrasonic probe to perform operations different from those of the 1D ultrasonic probe.

In the case where a 2D ultrasonic probe is used for the observation of the carotid artery 31, it is preferable to generate echo data in a range of at least 25 mm² (5 mm in azimuth direction by 5 mm in a lens direction) because the size of the early lesion of arteriosclerosis is in a range approximately from 1 mm to 10 mm. The lens direction refers to a direction in which the acoustic lens, disposed at the front of the ultrasonic transducer, curves (a so-called elevation direction for the 1D ultrasonic probe). In FIG. 10A, the lens direction is the y direction. The azimuth direction (a so-called azimuth direction for the 1D ultrasonic probe) is a direction in which the ultrasonic transducers are arranged, and is vertical to the lens direction. In FIG. 10A, the azimuth direction is the x direction. Even if the designated ROI is smaller than 25 mm², it is preferable that the 2D ultrasonic probe generates the echo data in a range of at least 25 mm² (5 mm in azimuth direction ×5 mm in a lens direction) containing the designated ROI as with the 1D ultrasonic probe. In this case, it is preferable that the number N of transmission of ultrasonic beams be equal to or less than “58”.

In the above embodiment, the strain ε is calculated as an example of the elasticity index. Alternatively or in addition, another elasticity index or elasticity data, for example, a strain rate, a stiffness parameter β, an elastic modulus, or the like can be calculated to generate an elasticity index image 53 showing the calculated elasticity index. For example, an elastic modulus Er in the diameter direction is calculated using a mathematical expression Er=Δp/ε where Δp represents a difference between the systolic blood pressure (SBP) or maximum blood pressure and diastolic blood pressure (DBP) or minimum blood pressure. An elastic modulus Ee in the circumferential direction is calculated using a mathematical expression Eθ=1/2×(r/h+1)×Δp/ε where “r” represents an inner radius of a blood vessel during a telediastolic phase of the cardiac cycle, and “h” represents the thickness of the blood vessel wall. In the case where the elastic modulus Er or Eθ is displayed as the elasticity index, a difference Δp between the systolic blood pressure and diastolic blood pressure may be measured using a device (manometer) provided separately from the ultrasonic diagnostic apparatus 10. The measured values are input to the ultrasonic diagnostic apparatus 10 through the operation section 13. The manometer and the ultrasonic diagnostic apparatus 10 may be connected to automatically input the blood pressure of the patient.

In the above embodiment, the echo data of one scan line is generated per ultrasonic transducer 16 for the sake of convenience. Alternatively, the echo data of two or more scan lines may be generated per ultrasonic transducer 16.

In the above embodiment, the seven ultrasonic transducers 16 are driven per transmission of ultrasonic beams, and the echo data of 3 scan lines corresponding to 3 ultrasonic transducers 16 is generated from the echo. Any number of ultrasonic transducers 16 may be driven per transmission. Four or more scan lines of echo data may be generated per transmission of the ultrasonic beams.

In the above embodiment, as an example, the B mode image 41 is colored based on the strain ε to generate the elasticity index image 53. Alternatively or in addition, other images, for example, the M mode image 49, may be colored based on the strain ε to generate an elasticity index image.

In the above embodiment, the carotid artery 31 is observed as an example. The ultrasonic diagnostic apparatus 10 may be used for echocardiography. The ultrasonic diagnostic apparatus 10 may be used for observing other sites.

Various changes and modifications are possible in the present invention and may be understood to be within the present invention.

What is claimed is:

1. An ultrasonic diagnostic apparatus having multiple ultrasonic transducers, the ultrasonic transducers transmitting ultrasonic beams to an object of interest and receiving echo from the object of interest and converting the echo into echo signals, the ultrasonic diagnostic apparatus comprising:
a transmitter for forming L of the adjacent ultrasonic transducers into a group and inputting a drive signal to each of the ultrasonic transducers in the group to transmit the ultrasonic beams to the object of interest, the transmitter making the ultrasonic transducers to transmit the ultrasonic beams with shifting the group by P (L>P≧2) of the ultrasonic transducers;
a receiver for receiving the echo signals from the ultrasonic transducers;
an echo data generator for generating echo data for each scan line based on the echo signal, the echo data being data of the object of interest in a depth direction;
a memory for storing multiple frames of the echo data;
an elasticity index calculator for calculating an elasticity index of the object of interest based on the echo data; and
an image generator for generating an elasticity index image, the elasticity index image being a cross-sectional image of the object of interest based on the echo data and colored in accordance with the elasticity index.

2. The ultrasonic diagnostic apparatus of claim 1, further including a monitor for displaying the elasticity index image and a color map showing a relation between the elasticity index and a color.

3. The ultrasonic diagnostic apparatus of claim 1, wherein a number N of transmission cycles of the ultrasonic waves to acquire one frame of the echo data satisfies a mathematical expression (1) (1) N≦(1/FR)×(Vs×10²/2D)
where FR (unit: frame/s) represents a frame rate, Vs (unit: m/s) represents an average sound velocity (unit: m/s) in a human body, and D (unit: cm) represents a maximum depth of the object of interest.

4. The ultrasonic diagnostic apparatus of claim 3, wherein the number N is determined to satisfy the mathematical expression (1) in accordance with a width and a depth of a region of interest when the region of interest in which the elasticity index is to be calculated is designated in the object of interest.

5. The ultrasonic diagnostic apparatus of claim 3, wherein the number P is determined based on the number N.

6. The ultrasonic diagnostic apparatus of claim 3, wherein the frame rate FR is 100 or more.

7. The ultrasonic diagnostic apparatus of claim 3, wherein the one frame is produced with the number N of 58 or less.

8. The ultrasonic diagnostic apparatus of claim 1, wherein the ultrasonic transducers are arranged in two dimensions.

9. The ultrasonic diagnostic apparatus of claim 8, wherein the echo data is generated for an area of at least 5 mm in azimuth direction by at least 5 mm in a lens direction, and the number N is 58 or less.

10. The ultrasonic diagnostic apparatus of claim 8, wherein the object of interest is a blood vessel, and echo data relative to a cross-section passing through a center axis of the blood vessel is extracted along a center axis direction of the blood vessel, and the elasticity index inside the cross-section is calculated using the extracted echo data.

11. The ultrasonic diagnostic apparatus of claim 8, wherein the object of interest is a blood vessel, and when the blood vessel moves, a displacement amount of the blood vessel is calculated using the echo data; and the elasticity index is calculated using the multiple frames of echo data of a same cross-section read from the memory based on the displacement amount.

12. The ultrasonic diagnostic apparatus of claim 1, wherein the elasticity index is strain of the object of interest or a value calculated from strain.

13. The ultrasonic diagnostic apparatus of claim 1, wherein the elasticity index calculator determines multiple representative points in the depth direction of the object of interest in the echo data along at least one scan line in the frame, and identifies the representative points of the same scan line in another frame to track each representative point between the frames, and then a distance between the representative points is calculated for each frame, and strain between the representative points is calculated based on a maximum value of the calculated distance and a maximum change in the calculated distance.

14. A method for calculating an elasticity index comprising the steps of:
transmitting ultrasonic waves to an object of interest to obtain a first frame and a second frame sequentially;
determining multiple representative points along at least one scan line in the first frame in the depth direction of the object of interest;
identifying positions of the representative points along the same scan line in the second frame to track each representative point between the first and second frames;
calculating a distance between the representative points in each frame; and
calculating strain of the object of interest between the representative points as the elasticity index based on a maximum value of the distance between the representative points and a maximum change in the distance between the representative points.

15. The method of claim 14, further comprising the steps of:
coloring the first frame in accordance with magnitude of the strain to produce an elasticity index image; and
displaying the elasticity index image and a color map representing a relation between the magnitude of the strain and a color on a monitor.

16. The method of claim 15, wherein the object of interest is a blood vessel.
